(19) United States
(12) Patent Application Publication
(10) Pub. No.: US 2005/0222628 A1
(43) Pub. Date: Oct. 6, 2005

(54) Implantable device for pain control and other medical treatments

(76) Inventor: Alexander A. Krakousky, San Luis Obispo, CA (US)

Correspondence Address: JOHN R. ROSS, PO BOX 2138, DEL MAR, CA, 92014, US

(21) Appl. No.: 10/980,911

(22) Filed: Nov. 4, 2004

Related U.S. Application Data
(63) Continuation-in-part of application No. 10/813,741, filed on Mar. 31, 2004.

(51) Int. Cl. A61N 1/18

(52) U.S. Cl. 607/3; 607/46

(57) ABSTRACT

A device for pain control or other medical treatments of a patient. The device includes an implantable unit for implantation under the skin of a patient. The implantable unit includes a drug chamber containing a fluid drug, a tube for delivering the fluid drug to a location in the patient's body, an electronic pump for pumping the drug through the tube to the location, a programmable processor for controlling the pump, and an electronic receiver for receiving control signals from outside the patient's body and delivering the control signals to the processor. The device also includes a controller located outside the patient's body for transmitting control signals to said receiver. In preferred embodiments the implantable device also comprises an electrical pulse generator and at least one electrode implanted at a location in said patient's body and connected to said pulse generator wherein said processor is programmed to control said pulse generator to deliver electrical pulsed to said electrode. In a preferred embodiment for the control of pain, the implantable chamber contains a refillable pain-relieving drug that is placed inside the chamber.

This application is a continuation in part application of Ser. No. 10/813,741. This invention relates to medical devices and methods and particularly to implantable devices and the use of those devices methods for pain control and other treatments.

### BACKGROUND OF THE INVENTION

### Pain Problems

Low back pain affects 40 -50 millions people and is the second most frequent cause of attendance at physician offices in the United States. Pain is responsible for work place absenteeism, reducing productivity and as a result is a major economic problem. Low back pain affects male more often then female. The major reason to develop back or neck pain is degenerative disks disease or disk herniation. Surgery is often attempted to relieve chronic back or neck pain, but these surgeries are often unsuccessful and acute pain syndrome transferred to chronic pain syndrome. For example, laminectomy (the excision of vertebral lamina) and discectomy (the excision of vertebral disks) as treatments of low back or neck pain are very common surgical procedures that can become a cause of the pain itself. Acute pain can be treated medically but chronic pain can not. Electrical stimulation of the spinal cord in specifically designated areas can successfully treat chronic intractable pain.

Pain in pelvic area in humans is a serious problem and it is tremendously underreported. The vast majority of the patients with pelvic pain are female. Many do not complain until the pain becomes unbearable. After the pain sensations have advanced from acute stage to chronic stage, hope for the cure of the pain often disappears. Patients become chronic pain patients with constant unrelieved pain. Sometimes pain accompanies spasticity problems that require additional treatment. One of the more specific pelvic pains relates to interstitial cystitis pain for which there is no effective treatment today. For these types of patient regular spinal cord stimulation in lumbar, sacral and pelvic areas has been attempted for medical management of the pain with some limited success. Intrathecal pump implantation represents the last resort for patients with chronic intractable pain syndrome.

### Sex Problems

Sexual performance in humans as well as in higher animals involves many functions. In males, there is erection, emission, ejaculation and orgasm. In females, there is initiation of sexual desire, escalation of the desire and orgasm. A wide variety of medical and psychological problems could interfere with one or more of these functions. The inability to achieve an erection is referred to as an erectile dysfunction or impotency. The inability to achieve an orgasm in females is referred to as anorgasmy. The principal methods presently used for male impotence correction and treatments include psychological and pharmacological treatments. Pharmacological treatments include noninvasive treatment (pills) and interventional treatment, which includes injection of vasoactive drugs into the penis. Surgical correction of impotence also exists. There is plastic surgery, prosthetic implantation and penile augmentation. There is no medical or surgical treatment that exists for anorgasmy in females at this time.

### The Spinal Column and the Spinal Cord

The central nervous system in humans is comprised of the brain and the spinal cord. Nerve fibers running within the spinal cord provide communication between the brain and various parts of the body. Some actions (reflex actions) are mediated through nerve connections in the spinal cord without involving the brain. Nerves carrying signals to the central nervous system are called afferent neurons and nerves carrying signals away from the central nervous system are called efferent neurons. The spinal cord is contained within the spinal column (also called the vertebral column). The spinal column is comprised of 26 irregular bones connected into a flexible curved structure. These are grouped into five sections. From the top down these five sections are the cervical curvature with 7 vertebrae, the thoracic curvature with 12 vertebrae, the lumbar curvature with 5 vertebrae, the sacrum with 5 fused vertebrae and the coccyx with 4 fused vertebrae. C, T, L, and S numbers (i.e., C1 through S5, numbered from the top of the cervical curvature to the bottom of the sacrum) identifies locations along the spinal column. See FIG. 4A. The spinal cord runs down from the brain through more than half of the spinal column. It terminates in or near the top of the lumbar curvature. Some nerves providing communication with the lower parts of the body continue on down through the spinal column. These nerves include the lumbar spinal nerves. The spinal cord and the lumbar spinal nerves are protected within the spinal column by a tough sheath called the spinal dural sheath. Just external to the spinal dura is a rather large epidural space filled with fat and a network of veins. The fat forms a protective padding around the spinal cord. See FIG. 4B. The dura and the epidural space extend well beyond the end of the spinal cord. Nerves branch out from the spinal column throughout its length to serve the various sections of the body. For the most part separate sets of nerves are provided for the left and right sides of the body.

### How the Sex Organs Work

FIGS. 5 and 6 show reproductive features of a male human and a female human. Erection of the penis is generally a necessary prerequisite for penetration of the vagina. The stimuli for this reflex may involve: (1) the sacral segment of the spinal cord where the pudendal nerve is initiated, (2) peripheral pudendal nerve receptor stimulation (around the penis) or (3) mental stimulation. The stimuli exist in the forms of electrical signals. These signals are transmitted via nerves. The afferent signals are transmitted via the pelvic nerves (right or left side) to the sacral segment of the spinal cord. Efferent signals are transmitted via the pudendal nerves. The pudendal nerves provide electrical signals to the penis arteries and a very large number of small arteries inside the penis in corpora cavernosa and corpora spongiosa. These electrical signals result in dilatation of the arteries permitting an increase of blood flow into the penis, which has the effect of partially restricting the veins taking blood out of the penis. As a result, there is a rapid filling of the blood spaces in the corpora cavernosum and corpus spongiosum areas of the penis. The swelling erectile bodies within the penis press on blood vessels draining the penis slowing the drainage. This physiologic effect makes the penis rigid. Therefore, the net effect is erection. Emission is the movement of spermatozoa and secretions from the testes and other accessory glands into the urethra. This is entirely a reflex process not involving the brain. The afferent side of the reflex arc is initiated by touch receptors in the genital area such as a receptor in the gland penis. Electrical signals travels via the pelvic plexus, the sacral segment of the spinal cord and the pudendal nerve to stimulate sympathetic fibers, which stimulate the ductus deferens to slow pump sperm and seminal fluid into the urethra. Ejaculation is the propulsion of the semen out of the urethra. The same afferent paths are involved. Central connections are located in the sacral segment of the spinal cord. Afferent and efferent stimuli are conveyed by pars sympathetic fibers of the pelvic splenic nerves and the pudendal nerves. Ejaculation is caused by the rhythmic contraction of the bulbocavernosus muscle, while the internal vesicle sphincter closes, preventing retrograde ejaculation into the bladder.

### Prior Art Patents

U.S. Pat. Nos. 5,246,015; 5,065,744 and 4,869,241 provide mechanical support for producing an erection. U.S. Pat. Nos. 5,236,904; 5,256,652 and 5,236,904 are the pharmaceutical type of impotence correction providing drugs administered to the penis. U.S. Pat. No. 5,454,840 issued to Applicant and one other describes a device and method for impotence correction. An electronic device is implanted inside the body. It is programmable and controllable from outside the body. The press of a button sends an electronic signal that initiates a process that simulates the body's natural reproductive processes. In a preferred embodiment, a programmable electronic device is implanted under abdominal muscle rectus. An electrical conductor is stitched to the surface of the pelvic splanchnic nerve. Stimulation of this nerve by a series of low voltage electrical pulses from the electronic device causes dilation of the penis arteries which results in an erection. The electronic device is controlled by a controller operated by the patient or his partner. Patent No. '840 is incorporated herein by reference.

What is needed is an improved device and method for correcting chronic intractable pain syndrome secondary to any possible causes of the dysfunction of spinal column, as well as correction of dysfunctional impotence that simulates the natural processes of erection and ejaculation as closely as feasible.

### SUMMARY OF THE INVENTION

The present invention provides a device for pain control or other medical treatments of a patient. The device includes an implantable unit for implantation under the skin of a patient. The implantable unit includes a drug chamber containing a fluid drug, a tube for delivering the fluid drug to a location in the patient's body, an electronic pump for pumping the drug through the tube to the location, a programmable processor for controlling the pump, and an electronic receiver for receiving control signals from outside the patient's body and delivering the control signals to the processor. The device also includes a controller located outside the patient's body for transmitting control signals to said receiver. In preferred embodiments the implantable device also comprises an electrical pulse generator and at least one electrode implanted at a location in said patient's body and connected to said pulse generator wherein said processor is programmed to control said pulse generator to deliver electrical pulsed to said electrode.

### Pain Control

In a preferred embodiment for the control of pain, the implantable chamber contains a refillable pain-relieving drug that is placed inside the chamber. The bottom of the chamber contains a piston with a coil and electromagnetic step driver. Programmable controls are provided to cause medication in the drug delivery container to be delivered to the body upon command initiated by the patient or his assistant. Pain can also be controlled by electronic stimulation using the stimulator referred to above. The stimulator is programmed to send a specific combination of amplitude, rate and pulse width that act through a specific set of electrodes. Upon command initiated by the user the stimulator produces a series of impulses that has been designed to subside the pain sensation. This embodiment works on both sexes males and females to treat chronic pain. In the preferred embodiment, the programmable electronic stimulator is implanted under the skin in the patient's back.

### Treatment of Sex Problems

Upon command initiated by the user or the user's lover the stimulator produces very short low-voltage electrical pulses in the sacrum section that are picked up by the nerves leading to the sex organs of the user, which stimulates arousal in the user's reproductive systems. The pulses are similar to the pulses generated by heart pacemakers. The present invention works on both males and females. In a preferred embodiment, the programmable electronic stimulator is implanted under the skin in the patient's back. Stimulation of the nerves coming out from the parasympathetic part of the spinal cord causes dilatation of the penile arteries in the male and in the clitoris arteries of the female, which results in an erection in the male and pre-orgasmic sensation in the female. In female, the stimulation of the sacral part of the spinal cord increases sexual desire and escalation to the level of orgasm. A preferred embodiment provides for emission stimulation. Emission is stimulated by electrical excitation of the sacral part of the spinal cord by increasing the voltage of the previous impulses. The device may be preprogrammed to set in motion the emission and ejaculation process at a predetermined time interval after the start of the erection process. The controller also can be programmed to permit the patient or his/her partner to initiate the emission and ejaculation process. In the third preferred embodiment, a drug is administered from a stored chamber in the device. The drug is transmitted via a tube inside the spinal canal. As with the first two embodiments, control is in the hands of the user or the user's lover. Various combinations of the described above embodiments represent additional embodiments of the present invention.

### BRIEF DESCRIPTION OF DRAWINGS

FIG. 1 shows a preferred embodiment installed in a user.

FIGS. 2A and 2B show the components of a preferred embodiment.

FIGS. 3A and 3B show preferred pulse shapes.

FIGS. 4A and 4B show features of a human spinal column and spinal cord.

FIGS. 5 and 5A and 6 show additional preferred embodiments of the present invention.

FIG. 7 and 7A show features of a device for pain control and other medical treatment.

### DETAILED DESCRIPTION OF PREFERRED EMBODIMENTS

### Treatment of Sex Problems

### First Preferred Embodiment

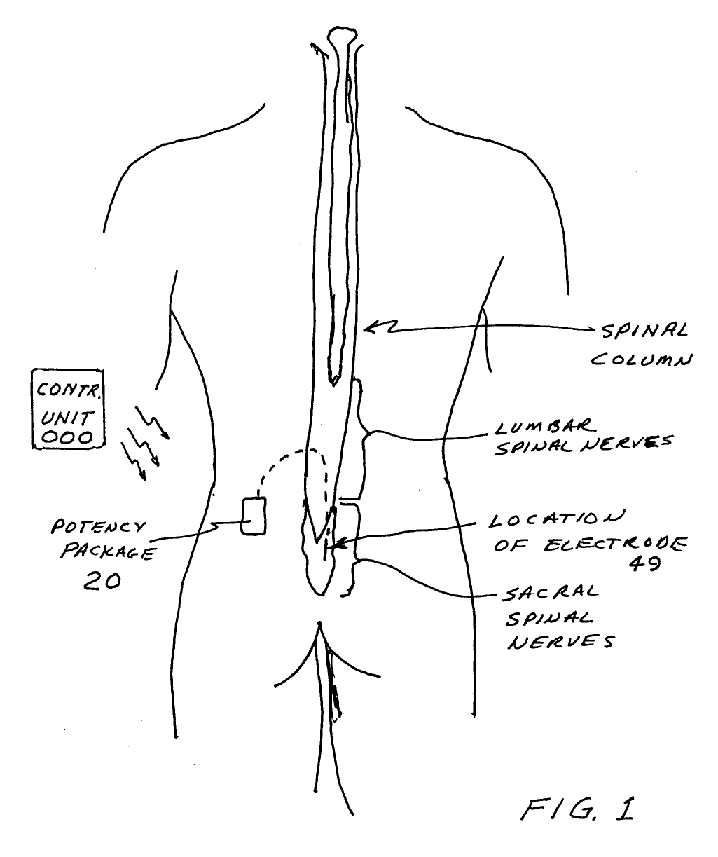

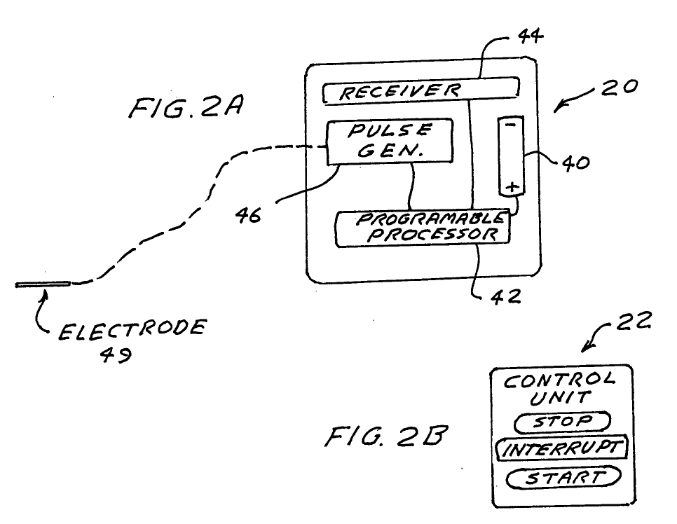

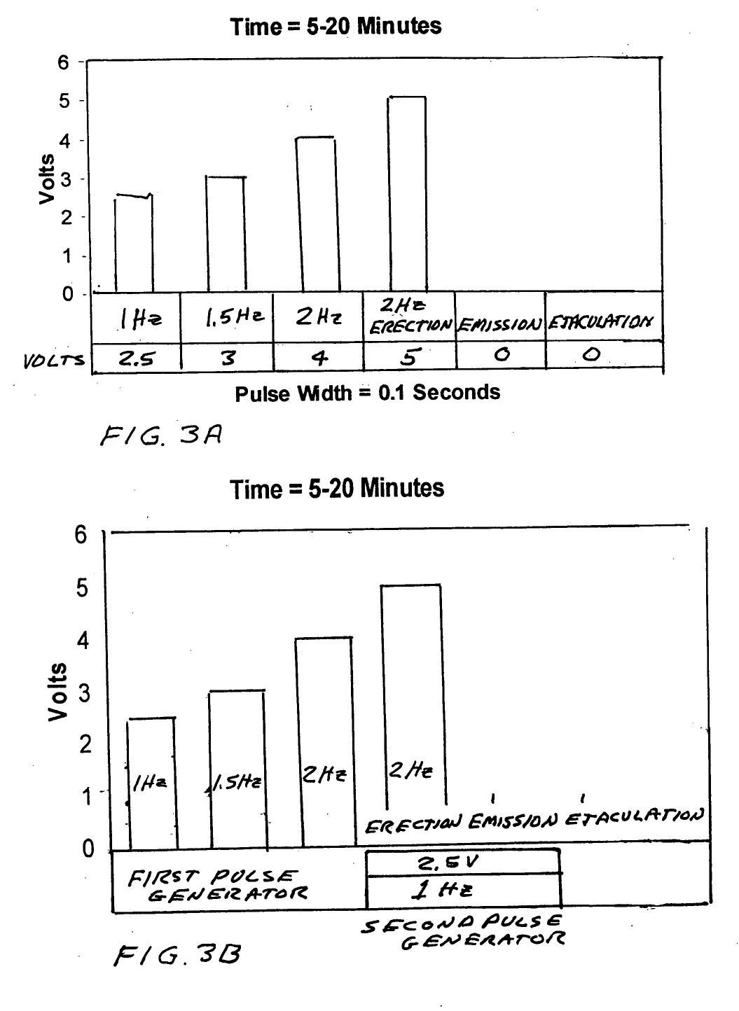

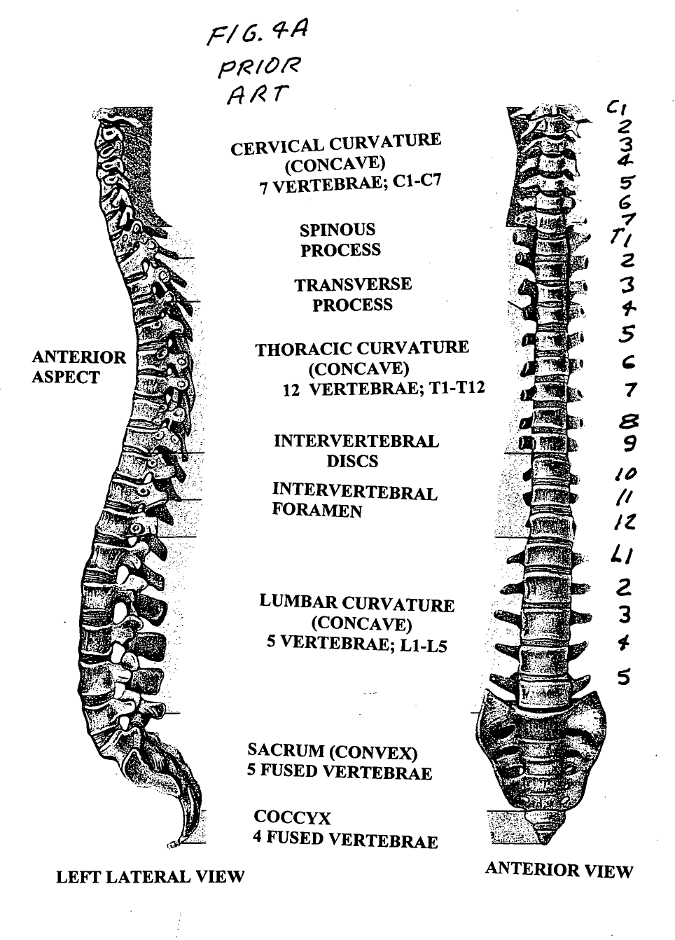

FIG. 1 shows a preferred embodiment of the present invention. A two-inch long thin electrode 49 located in the epidural space at the S2, S3 and S4 level of the sacrum on the right side of the sacral spinal nerves is connected by an electrical conductor running under the skin of the user to a stimulator device 20 implanted under the skin of the user in the top part of the user's buttox. FIG. 2A is a block diagram of the stimulator device, which the Applicant calls Potency Package Two. A preferred prototype embodiment comprises a modified commercial pacemaker Model 600AV manufactured by Siemens and modified by the Applicant. The unit comprises a battery 40, a programmable signal circuit 42, a pulse generator 46 and a receiver antenna 44. The unit is controlled with an external control unit 22 shown at FIG. 2B. The unit comprises a start button, stop button and an interrupt button. The preferred sequence of pulses that should provide good results for many patients is shown in FIGS. 3A and 3B. The package can be reprogrammed to change any of the parameters shown in FIGS. 3A and 3B, which are pulse height, pulse width, frequency, duration and sequence. The best program for each individual patient can be only determined by testing. These parameters such as number of pulses grouped, voltage, rates and pulse duration are well within the range available with the above potency package device. These parameters are among others that could be programmed, with the range of the device, using a commercially available pacemaker programmer such as Model #3CMHK850 supplied by MIFI and SMHK and also described in patent '015 referred to in the Background Section. The programmer transmits programmed information via a pulse site and magnetic field generated in the programmer, to the electromagnetic detector in a programmable signal circuit of the implanted device. This device, shown in FIG. 2A, comprises only one electrode. Before permanent implantation of the device, every patient has to go through the testing trial. The trial designed to detect the ability of the stimulation to achieve an erection. The 2-inch long electrode runs from the epidural space at level S2, S3 and S4 of the sacral segment to under the patient's skin to connect the device. The procedure can be accomplished in any surgical center under monitored anesthesia care and local anesthesia. Personal surgical skill is required in order to install the device. The recommended electronic pulse series is shown in the FIG. 3A. If this series does not provide the desired effect, the doctor can vary the parameters. If an erection is produced by any of the tests, then the doctor continues the process and permanently installs the Potency Package Two as described above. If the doctor is unable to produce an erection in three days trial period, the doctor may choose not to proceed with the permanent implantation. In women patients, the clinical trial of the orgasm initiation stimulation contains the same approach in the same segment area in the sacral part of the spinal cord as in male patients. Instead of erection the doctor will be testing for pre-orgasm stimulation.

### Surgery

The details of the surgery designed to provide the implantation of the electrode and device as described above is now described. A trained surgeon should surgically implant the Potency Package Two device and electrodes. The operation is very similar to the implantation of the heart pacemaker. The patient should be previously anesthetized, spontaneously breathing with the application of standard monitoring by American Society of Anesthesiology, which includes EKG, blood pressure, pulse monitor and oxygen by nasal cannula. The patient should be put in the prone position on the operating table and the site on the lumbar part of the patient's spine should be prepped and draped in sterile fashion. Local anesthesia should be applied to the lower lumbar area, and a 2-cm incision of the skin in the middle of the spine should be performed. The epidural space should be identified using loss of resistance technique with a Tuohy needle and fluoroscopic imaging. When the epidural space on the left side is identified, the electrode should be transmitted through the needle, and the needle should be withdrawn. The electrodes should enter to the epidural space at the level of lumbar part of the spinal cord and advance down to the sacral region. The proper position o f t he electrode should be verified under fluoroscopy. The electrode should be advanced to the sacral segment of the spinal cord at the level of S2, S3 and S4 on the left side. If an electrode is to be applied to the right side instead of or in addition to the left side the same loss or resistance technique and fluoroscopic imaging has to be applied on the right side of the spine to identify the epidural space on the right side. The right-side electrode should be positioning on the same level on the right side of the patient's sacral segment, S2, S3 and S4. The proper position of the second electrode should be confirmed by the fluoroscopy. After the proper positioning of the electrode (or electrodes) on the side or sides of the patient's spinal nerves in the epidural space at the level of the sacral segment of the spinal cord, a voltage signal (at a low range of available voltages) with the proper setting of the impulses should be applied through the electrode to the spinal cord. The patient should be asked what kind of sensation in his or her genitalia has been felt. If the male patient has established the initiation of the erection process, the electrodes should be affixed at those levels. If the initiation of the orgasm in the female has been identified by electrical stimulation, the electrodes also have to affix and sutured at the level, which was identified.

The stimulator should be installed in the buttock area just below the waist on the left or on the right side as the patient prefers, a 5-cm incision should be made after application of local anesthesia to this area. The pocket for the pacemaker should be made there. The tunnel from the initial part where the electrode has entered to the patient's body at the level of the lumbar part of the spinal cord should be transmitted to the pocket area. By using a screwdriver, the electrode is connected to the stimulator, and the skin over the incisions should be closed.

### Second Preferred Embodiment

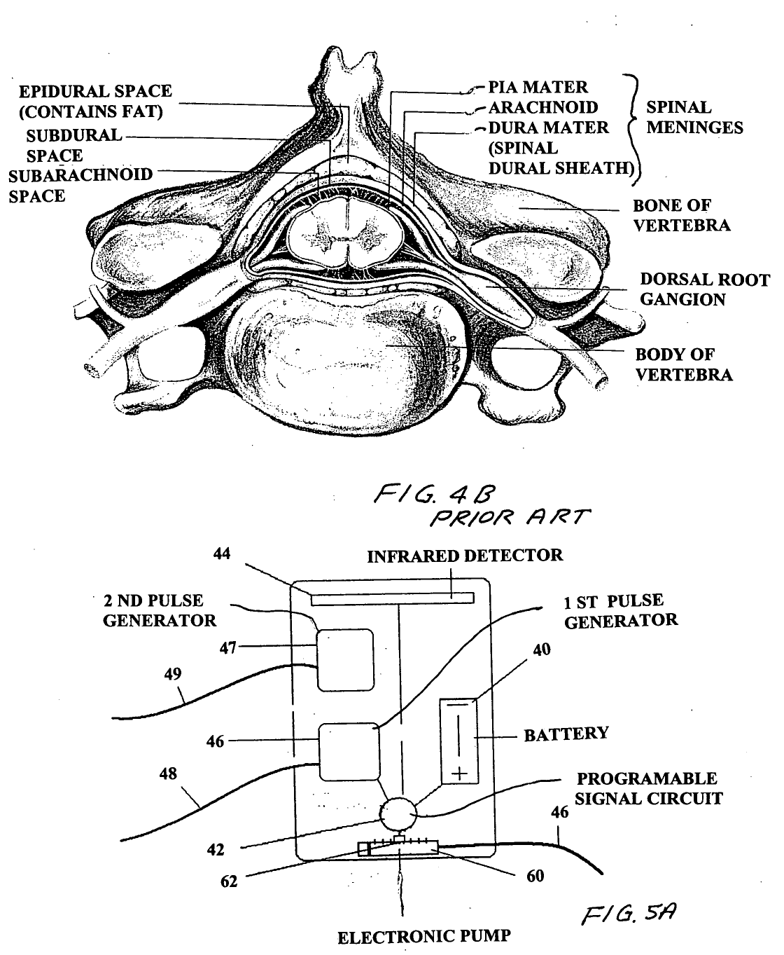

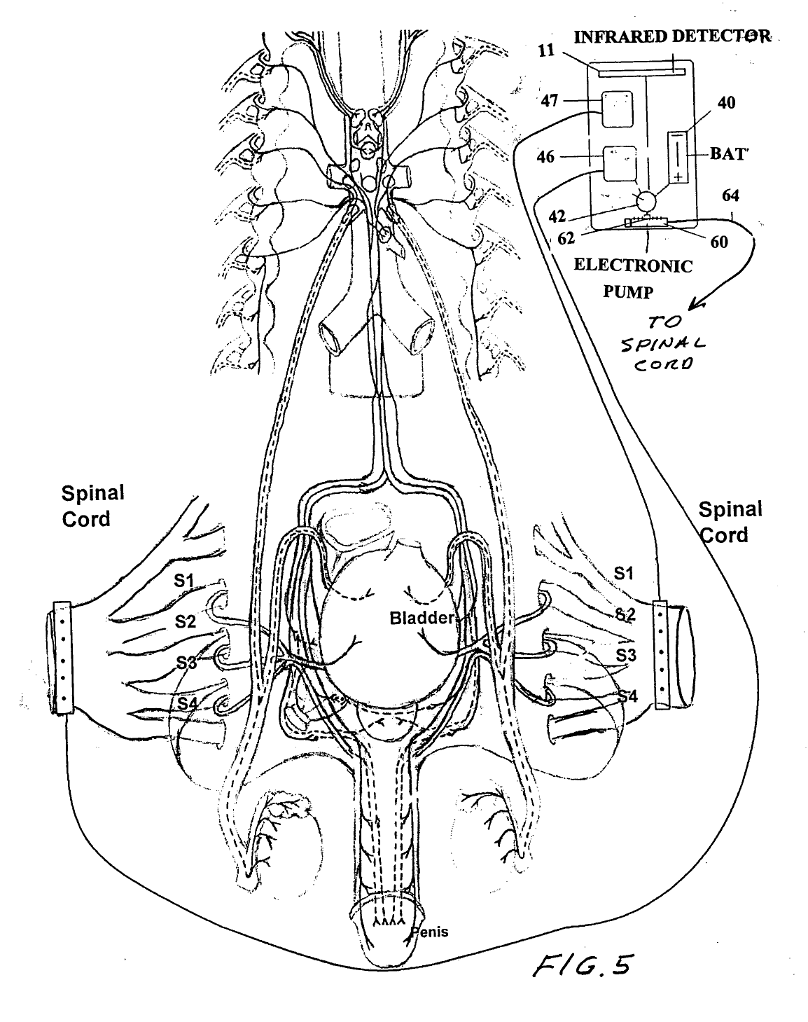

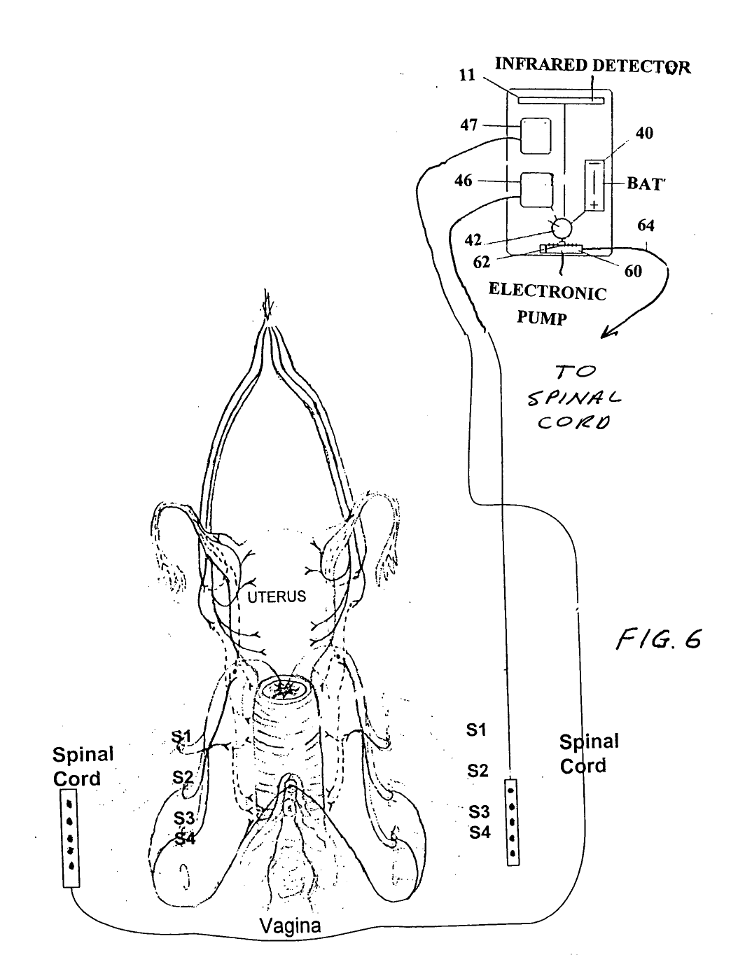

In a second preferred embodiment shown in FIG. 5, the stimulator contains a chamber 60 for storage within the body of a drug such papaverine and a small electronic pump 62 and a very thin tube for delivering of the drug to the spinal canal. The same result will be achieved with the delivering of the drug to the patient's body through the tube placed inside the spinal canal and deliver the drug in an on-and-off fashion to initiate the erection in the male. The delivery of the drug is initiated by an electronic signal transmitted by a hand-held transmitter controlled by the patient. For these alternative two electronic circuits are programmed as described above. The controller is programmed to deliver the drug at the time 0. A drug delivery chamber consists of plastic refillable containers, which is placed into hermetic chamber 62 as shown in FIG. 5A. The bottom of the chamber is a piston with a coil and electromagnetic step driver. The first step of the erection stimulation is a vasoactive drug (such as papaverine) delivered by sending an electrical potential to the driver. The driver pulls the coil into the electromagnet to apply a force squeezing the drug into the tube attached to the plastic container at one end and implanted into the spinal canal at the other end of the tube. Chamber 60 is refillable upon injection through the skin with a syringe injector. FIG. 6 shows this second embodiment as it is applied to a female.

### Equipment

The potency package components can be standard off-the-shelf components. The components include: lithium battery, LBSAR5, made by SARATOF with a life-time of five to eight years, a pulse generator CLG445 made by MIFA, a receiver/transmitter MC145027 made by Motorola and IR remote control receiver 2338M made by AEG Corporation, and a fast IR Protodiode detector S113-11 made by Hamamatsu, IR remote control transmitter U327-M made by AEG Corporation, stepping motor 155 ML microslide made Toshiba Corporation, Silicon tube catheter T5715 made by Dow Coming Silastic and elastimer Q7-4750 Silicon pack made by Dow Coming Silastic.

### Other Embodiments

Various combinations of the above-described embodiments provide many other embodiments of the present invention. The fourth embodiment would provide for emission stimulation only. A fifth is drug only. The following table lists vasoactive drugs and recommended quantities.

| | |
|---|---|
| Papaverine | 15 mg |
| Phentolamine | 0.5 mg |
| Prostaglandin E1 | 20 mcg |
| Vasoactive intestinal polypeptide | 5 mcg |

Providing more than one drug will design in additional embodiment. For example, a drug such as nitroglycerin releasing into the blood stream to providing protection for patients against heart attack during sex. The following table shows some drugs recommended for correction of the most common health conditions occurring during sexual intercourse.

| | |
|---|---|
| Arrhythmia | beta-blockers |
| Asthma | alpha-blockers |
| Angina | nitroglycerin |
| Hypertension | beta-blockers |

Using many different drugs that are known to induce erection provides additional embodiments. Also, there are many electronic pulse sequences, which would work well to produce erection, emission and ejaculation for many different patients in addition to the sequences described above. Skilled doctors will recognize that electrodes can be connected at different locations other than those described above. To correct the arterial circulatory problem of impotence, the arterial anastomosis should be performed at the same time with the Potency Pack Two implantation. Also the penis enlargement surgery and the penis lengthening surgery could be applied at the same time as the implementation of the electrodes for the correction of male impotence. The vascular problems related to peripheral vascular disease should preferably be treated with anastomosis between inferior hypogastric arteries and the central and dorsal artery and dorsal vein of the penis.

### Diabetic Type Impotency Correction

To correct the diabetic circulatory problem of impotence, the arterial anastomosis should be performed to the penis at the same time with the Potency Package Two implantation. Anastomosis between the hypo-gastric artery and dorsal artery should be performed end-to-end or end-to-side. The penile vein of diabetic patients can usually provide blood flow and surgical correction is not required in most cases.

### Male Enhancement Surgery

Potency Package Two could be implanted during the period of penis enlargement by Dermograph, or by Allograft. They are implanted inside the penis with the preservation of the corpora cavernosum and the corpus spongiosum. The dermal graft or allograft is implanted under the skin of the penis with fixation to the distal part of the glands penis and the proximal part of the shaft of the penis. The penile lengthening is done by dissection of the tendon, which is affixed to the base of the penis and the pubic bone. Increase in length of the penis usually is from 1½ inches to 3⅓ inches. Increase in girth of the penis depends upon the patient's preference. The average increase in penis size is 2½ inches to 4½ inches.

### Treatment of Anorgasmia in Females

The surgical technique described above for males can be applied to treat anorgasmy in women. The stimulation of the sacral part of the spinal cord generates impulses through the pudendal nerve that supplies innervation to the inner and outer part of the vagina and the clitoris. This artificial stimulation leads to the generation of the impulses that provide the additional blood supply and lubrication to the vagina and increase of the arousal part of the sexual desire in women. The additional stimulation technique applied through the nervous system allows women to feel a higher level of sexual arousal, increase escalation of the orgasm and the actual achievement of the orgasm. The higher level of sexual arousal increases the ability to achieve orgasm in the majority of human subjects.

### Animal Use

The present invention can be applied to many animals. It should be especially valuable for use with breeding animals such as prize bulls. It could also be used in a breeding program of captured members of endangered species in wild animals.

### Diabetes Problems

Insulin dependent diabetes is one of significant health problem that present a cause of death in million people worldwide. The implantable pump described above may be used to provide insulin when needed. The drug container, chamber 60 in FIG. 5A could contain any types of insulin for constant control of patient's blood sugar and inject the medication under the skin. The implantable device will be equipped with a sugar control detector that could be set for certain level of the patient's blood sugar and automatically deliver the certain amount of medication related to the quantity of blood sugar.

### Chronic Seizure Disorders and Parkinson Problems

Chronic seizure disorders and Parkinson disease represents unsolved problems for medical practitioners. Many types of stimulation were used to treat seizer disorders, but result was unsuccessful. Implantable drug chamber can control Parkinson disease tremors as well as generalized seizures with constant level of medication that will injected inside the human body by the implantable pump though the catheter that could be implanted under the skin or inside the intrathecal space. Chronic seizure disorder could be predictable and could be unpredictable. Patients with chronic seizure disorders have to have a constant level of anti-seizure medication. The half-life of antiseizure medication pills is short and requires constant addition of supplement medication several times a day. Existing of medication deposited inside the patient's body will help to prevent the occurring of the seizure. The medication depot could be located under the skin or injected under constant condition inside the intrathecal space. In the case of Parkinson disease, the constant hand shaking tremendously affected patient's general life stile. Constant level of injectable medication from the implantable drug chamber either under the skin or inside the intrathecal space, will normalize neurotransmission or prevent hands from shaking.

### Chronic Spasticity Problems

Many patients after spinal cord injury suffer from generalized spasticity disorder. There are many varieties of medication used to control chronic spasticity disorders. The implantable pump showed in FIG. 5A represents a solution for these patients. Constant delivery of the medications to the spinal canal or under the skin represents one of the possibilities to control constant spastic muscle contraction. Chronic spasticity is a disorder when proper communication between nervous fibers and muscular fibers destroyed by spinal cord trauma, congenital malformation or acquired disease. Therefore, the only logical choice to take care of the spasticity problem is to relax the muscles. Constant muscle contractions are painful and require serious attention. The existing of certain level of antispastic medication will help with pain sensation and relax the muscles. None of the existing oral medications can do this because they are not potent enough or they developed side effects. Medication's deposited under the skin or inside the intrathecal space will allow muscles to relax and prevent patient form constant pain sensation.

### Pain Control

### The Implantable Drug Chamber for Pain Controls

As described above a preferred drug chamber comprises an electronic pump and very thin plastic tube for the delivering of the drug supply the body with the medication. The tube runs under the skin anywhere from the implantable pump with patient preference side of implantation. Also, the potency of the delivering of the medications will dramatically increase if the medication will deliver inside the intrathecal space. The delivery of the drug is initiated by the electronic signal that transmitted by a hand held transmitter within control of the physician or physician assistant.

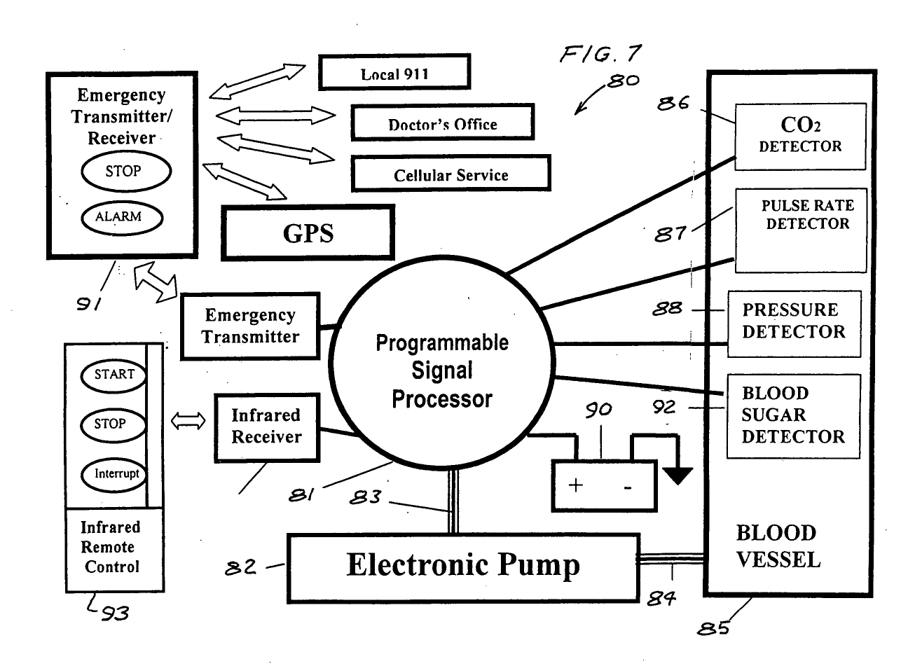

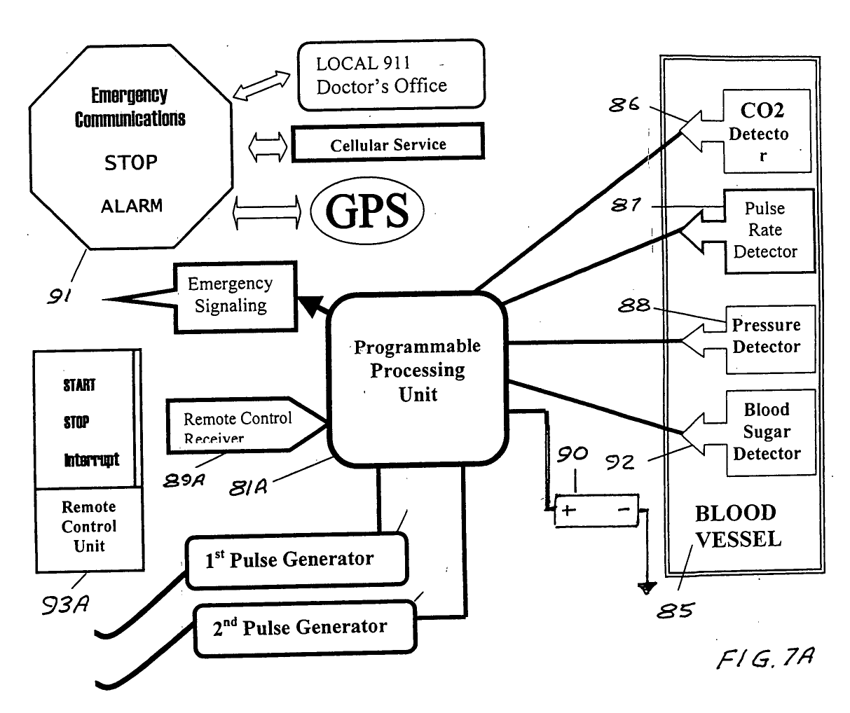

In some preferred embodiments, drug delivery is provided with no provision for electrical stimulation of nerves. FIG. 7 shows a programmable implantable drug delivery unit 80 containing no electrical stimulation of nerves. This embodiment contains two electronic circuits. A programmer 93 outside the body transmits programming information via an infrared transmitter (or a pulsating magnetic field) to a receiver in the programmable part of the implanted drug delivery unit 80. The unit comprises pump 82, infrared receiver 98, and battery 90. (Battery 90 may be a rechargeable battery that is charged through the patients' skin. This may be accomplished with a conductor injected through the skin to an electrical port on unit 80. Other more elaborate techniques for recharging the battery can be adapted for this purpose.) The unit may also be equipped with various sensors to provide information that can be utilized for automatic control of drug injection. These could include CO2 detector 86, pulse rate detector 87, blood pressure detector 88 and blood sugar detector 92. The drug delivery chamber contains a plastic refillable container that is placed into hermetic chamber. The bottom of the chamber contains a piston with a coil and electromagnetic step driver. There are several sequences of the events that require to be performed in order to deliver the medication to the body. As the first step of the delivering of the medication the electronic signal has to be sent to the driver. The driver pulls the coil into the electromagnet up to a definite programmed distance squeezing the liquid medication into the tube attached to the plastic container with one end and implanted under the skin at the other end of the tube. Also, the other end of the plastic tube could be implanted inside the intrathecal space, rather then under the skin according to the preference of the patient and the physician in order to increase the potency of the medications. The medication chamber is refillable by injection through the skin with a syringe injector. The volume of the plastic container could vary. At the beginning of the treatment the container could retain 20 cc of soluble medication, and after the treatment will be established it could be change to 40 cc container with second surgical procedure.

The drug chamber may be filled with opioids, local anesthetics or combination thereof for treatment of chronic intractable pain. In other embodiments the drug chamber may contain insulin or its derivatives to treat insulin dependent diabetes. Or the drug chamber may contain anti-seizure, anti-Parkinson or anti-spastic medication.

Surgery for Drug Delivery Device for Pain Control

The details of surgery designed to provide the implantation of the device as described above is now described. A trained surgeon should surgically implant the FIG. 7 device. The patient should be previously anesthetized, spontaneously breathing with the application of standard monitoring by American Society of Anesthesiology, which includes EKG, blood pressure, pulse monitor and oxygen by nasal cannula. The patient should be put in the prone position on the operating table and the site on the lumbar part of the patient's spine should be prepped and draped in sterile fashion. Local anesthesia should be applied to the lower lumbar area, and a 2-cm incision of the skin in the middle of the spine should be performed. The intrathecal space should be identified using spinal needle and fluoroscopic imaging. The conformation of the proper position of the needle is detected by the spinal fluid coming out from the needle. The outer part of the plastic tube will be positioned inside the spinal canal and inner part of the plastic tube will be connected to the pump. The pocket for the pump will be made under the skin in patient's abdomen in order to easier refill the pump with the medication. In case of the delivering the medication not into the spinal canal but just under the skin, the outer part of the plastic tube will be positioned in the abdomen area, close to the implantable pump.

The delivery of the drug is initiated by an electronic signal transmitted by a hand-held transmitter controlled by the physician or physician assistant. For these alternative two electronic circuits are programmed as described above. The controller is programmed to deliver the drug. As in the previously described embodiments, the drug delivery chamber consists of plastic refillable containers. The bottom of the chamber is a piston with a coil and electromagnetic step driver.

### Implantable Electronic Stimulator for Pain Control

Preferred embodiments of the present invention provide utilizes electronic stimulation for pain control in pelvic, lumbar, neck and limbs areas. Components for providing this stimulation are shown in FIG. 7A. An electronic stimulator with at least one pulse generator is implanted inside the body. This device is similar to the device shown in FIGS. 2A and 2B. At least one electrode is installed in the epidural space in the sacrum, lumbar or neck section of the spinal column and a conductor running under the user's skin electrically connects the electrode to the pulse generator. The stimulator is programmable and may be controlled from outside the body. The stimulation parameters delivering are determined by the programmer. Programmable parameters are amplitude (from 0 to 10 volts), pulse width (from 30 to 300 msec), mode (continuous or cycling), rate (from 1 to 100 Hz), and cycle (on/off). Various combinations of the described above fixtures represent additional embodiments of the present invention.

### Surgery for Electronic Pain Control

A two-inch long thin electrode is inserted in the epidural space at the L2, L3 and L4 level of the lumbar area on the right side of the lumbar spinal nerves. It is connected by an electrical conductor running under the skin of the user to a stimulator device implanted under the skin of the user in the top part of the user's buttock. The second electrode located on left side on L2, L3, and L4 areas. In the case of neck pain the electrodes apply on C2, C3, C4, C5, C6 or C7 levels on one side or on both sides depend upon the pain sensation level and quality. Control units for this purpose comprise a start button, stop button and an interrupt button as shown at 93A in FIG. 7A. The package can be reprogrammed to change any of the parameters which are pulse height, pulse width, frequency, duration and sequence. The best program for each individual patient can be only determined by testing. These parameters such as number of pulses grouped, voltage, rates and pulse duration are well within the range available with the of the shelf pulse generating devices described above. These parameters are among others that could be programmed, with the range of the device, using a commercially available pacemaker programmer such as Model #3CMHK850 supplied by MIFI and SMHK and also described in patent '015 referred to in the Background Section. The programmer transmits programmed information via a pulse site and magnetic field generated in the programmer, to the electromagnetic detector in a programmable signal circuit of the implanted device. Before permanent implantation of the device, every patient has to go through the testing trial. The trial designed to detect the ability of the stimulation to achieve pain free sensation. The 2-inch long electrode runs from the epidural space at level L2, L3 and L4 of the lumbar segment to under the patient's skin to connect the device. The procedure can be accomplished in any surgical center under monitored anesthesia care and local anesthesia. Personal surgical skill is required in order to install the device. If this series does not provide the desired effect, the doctor can vary the parameters. If pain free sensation is produced by any of the tests, then the doctor continues the process and permanently installs the Potency Package Two as described above. If the doctor is unable to achieve pin free sensation in three days trial period, the doctor may choose not to proceed with the permanent implantation.

After the proper positioning of the electrode (or electrodes) on the side or sides of the patient's spinal nerves in the epidural space at the level of the lumbar or cervical segments of the spinal cord, a voltage signal (at a low range of available voltages) with the proper setting of the impulses should be applied through the electrode to the spinal cord. The patient should be asked about the pain sensation in certain area (neck, back etc). If during the stimulation the pain sensation changed (subside or even disappeared) the electrode or electrodes should be left in that place. The stimulator should be installed in the same fashion that was described in original application.

### Detailed Surgical Procedure

The details of a preferred surgery procedure designed to provide the implantation of the electrode and device as described above is now described. A trained surgeon should surgically implant the device and electrodes. The operation is very similar to the implantation of the heart pacemaker. The patient should be previously anesthetized, spontaneously breathing with the application of standard monitoring by American Society of Anesthesiology, which includes EKG, blood pressure, pulse monitor and oxygen by nasal cannula. The patient should be put in the prone position on the operating table and the site on the lumbar part of the patient's spine should be prepped and draped in sterile fashion. Local anesthesia should be applied to the lower lumbar area, and a 2-cm incision of the skin in the middle of the spine should be performed. The epidural space should be identified using loss of resistance technique with a Tuohy needle and fluoroscopic imaging. When the epidural space on the left side is identified, the electrode should be transmitted through the needle, and the needle should be withdrawn. The electrodes should enter to the epidural space at the level of lumbar part of the spinal cord and advance down to the sacral region. The proper position of the electrode should be verified under fluoroscopy. The electrode should be advanced to the sacral segment of the spinal cord at the level of L2, L3 and L4 on the left side. If an electrode is to be applied to the right side instead of or in addition to the left side the same loss or resistance technique and fluoroscopic imaging has to be applied on the right side of the spine to identify the epidural space on the right side. The right-side electrode should be positioning on the same level on the right side of the patient's sacral segment, L2, L3 and L4. The proper position of the second electrode should be confirmed by the fluoroscopy. After the proper positioning of the electrode (or electrodes) on the side or sides of the patient's spinal nerves in the epidural space at the level of the sacral segment of the spinal cord, a voltage signal (at a low range of available voltages) with the proper setting of the impulses should be applied through the electrode to the spinal cord. The patient should be asked about the pain sensation in pelvic area. If during the stimulation the pain sensation changed (subside or even disappeared) the electrode or electrodes should be left in that place.

The stimulator should be installed in the buttock area just below the waist on the left or on the right side, as the patient prefers. 5-cm incision should be made after application of local anesthesia to this area. The pocket for the pacemaker should be made there. The tunnel from the initial part where the electrode has entered to the patient's body at the level of the lumbar part of the spinal cord should be transmitted to the pocket area. By using a screwdriver, the electrode is connected to the stimulator, and the skin over the incisions should be closed.

Another preferred embodiment describes the treatment of the pain in the back, the neck and the limbs. The electrodes and the pacemaker are positioning in the same epidural space of the human body, but in the different level of the spinal cord. For the treatment of lower back and lower extremities pain, the electrodes should position at the lumbar epidural space of the spinal cord. For the treatment of the neck and arms pain, the electrode should position in the neck epidural space of the spinal cord. For second preferred embodiment surgical incision has to be made in lumbar or cervical areas accordingly.

The present invention works on both sexes males and females to treat chronic pain syndrome in cervical, lumbar, pelvic or any other places in human body. Programmable parameters are amplitude (from 0 to 10 volts), pulse width (from 30 to 300 msec), mode (continuous or cycling), rate (from 1 to 100 Hz), and cycle (on/off). Various combinations of the described above fixtures represent additional embodiments of the present invention. The implantable stimulator send the signal to the leads that implanted inside the cervical, lumbar or any other epidural space in one end and the other end connected to the stimulator implanted under the skin in different areas of the patient's body after successful trial of the stimulation.

The stimulator could be a single-chamber device programmable mode device or it could be dual-chamber programmable mode device. In single chamber mode, the device sends the electrical signal to the electrode (wire) that located in one side of the spinal cord. In dual chamber mode the device will sends the electrical signal to two electrodes located in both sides of the spinal cord. A physician's programmer and patient's remote control regulate the operation of the stimulator. The implanted stimulator is operated by battery and directed by the electronic circuit that sends electrical impulses to implanted leads. The leads propagate the electrical impulses from the stimulator to the nerve structures in the dorsal aspect of the spinal cord. Over activation of these structures produce nerve impulses that inhibit the sensation and transmission if the pain.

While the above description contains much specificity, the reader should not construe this as limitations of the scope of the invention, but merely as exemplification of preferred embodiments thereof. This skilled art will envision many other possible variations like another location for the stimulator, different types of electrodes and pacemakers, different voltages, amplitudes, pulse groups, repetition rate, pulse duration, remote control with more or less functions, fully automatic preprogrammed pacemaker without external controls, etc. Additional electronic devices could be added to the potency package that contains implantable electrical stimulator, implantable intrathecal pump or combination of these two will be: 1. Heart rate monitor. 2. Blood pressure detector. 3. Blood sugar detector. 4. CO2 detector. 5. Emergency transmitter will send a signal to doctors' office, cellular service and GPS system. These devices also could be designed to produce an alarm if the patient's data indicated that he or she is becoming too excited in the course of sex so that the patient an his partner can moderate their love making. Accordingly the reader is requested to determine the scope of the invention by the appended claims and their legal equivalence and not by the examples, which have been given.

1. A device for pain control or other medical treatments of a patient comprising:
A) an implantable unit for implantation under the skin of a patient comprising:
1) a drug chamber containing a fluid drug,
2) a tube for delivering said fluid drug to a location in the patient's body,
3) an electronic pump for pumping said drug through said tube to said location,
4) a programmable processor for controlling said pump, and
5) an electronic receiver for receiving control signals from outside the patient's body and delivering said control signals to said processor,
B) a controller located outside the patient's body for transmitting control signals to said receiver.

2. The device as in claim 1 wherein said implantable device also comprises an electrical pulse generator and at least one electrode implanted at a location in said patient's body and connected to said pulse generator wherein said processor is programmed to control said pulse generator to deliver electrical pulsed to said electrode.

3. The device as in claim 1 wherein said device is programmed for pain control.

4. The device s in claim 2 wherein said device is programmed for pain control.

5. The device as in claim 1 wherein said device is programmed for treating diabetes.

6. The device as in claim 1 wherein said device is programmed for treating chronic seizure disorders.

7. The device as in claim 1 wherein said device is programmed for treating Parkinson disease problems.

8. The device as in claim 1 wherein said device is programmed for treating spasticity problems

9. The device as in claim 1 wherein said device is programmed for treating sexual problems.

10. The device as in claim 2 wherein said device is programmed for treating diabetes.

11. The device as in claim 2 wherein said device is programmed for treating chronic seizure disorders.

12. The device as in claim 2 wherein said device is programmed for treating Parkinson disease problems.

13. The device as in claim 2 wherein said device is programmed for treating spasticity problems

14. The device as in claim 2 wherein said device is programmed for treating sexual problems.
